(19) United States
(12) Patent Application Publication
(10) Pub. No.: US 2026/0207843 A1
(43) Pub. Date: Jul. 23, 2026

(54) MANUAL BOLUS VOLUME ESTIMATION

(71) Applicant: ICU Medical, Inc., San Clemente, CA (US)

(72) Inventors: Robert L. Hlinsky, Glenview, IL (US); James Jacobson, Lindenhurst, IL (US)

(21) Appl. No.: 19/135,951

(22) PCT Filed: Dec. 19, 2023
(86) PCT No.: PCT/US2023/084718

§ 371 Date: Jun. 5, 2025

Related U.S. Application Data
(60) Provisional application No. 63/433,679, filed on Dec. 19, 2022.

(51) Int. Cl. A61M 5/145; A61M 5/168

(52) U.S. Cl. CPC A61M 5/1456; A61M 5/1684; A61M 2205/103; A61M 2205/3379

(57) ABSTRACT

A syringe pump including a plunger driver head that selectively urges against a syringe. A drive arm includes an elongate plunger tube. The drive arm is coupled to the plunger driver head and movement of the elongate plunger tube corresponds with movement of the plunger of the syringe along the first central longitudinal axis. A potentiometer generates a signal that is proportional to an insertion distance of the rod inside the elongate plunger tube. The syringe pump records the signal from the potentiometer at the time delivery of infusate from by the syringe pump is paused and again at the time delivery is resumed. The syringe pump detects information about the syringe, accesses an electronic system to determine information about the syringe allowing the syringe pump to calculate the volume of a manual bolus from the syringe while delivery of infusate from the syringe pump was paused.

### CROSS REFERENCE TO RELATED APPLICATIONS

This application claims the benefit of Provisional Application No. 63/433,679 filed Dec. 19, 2022, the disclosure of which is hereby incorporated by reference.

### TECHNICAL FIELD

This disclosure relates generally to medical devices. More particularly, this disclosure relates to calculation of an estimate of a manual bolus administered while a syringe pump is powered on.

### BACKGROUND

In the field of medication delivery devices including so-called “syringe pumps,” typically a syringe is mechanically driven under computer or digital processor, or microprocessor (collectively, “processor”) control to deliver a prescribed volume or dose of a drug, fluid, fluid-like substance, or medicament (hereinafter, collectively, an “infusate”) at a controlled rate to a patient through an infusion line or tubing that is fluidly connected to the syringe. Syringe pumps typically include a motor that rotates a lead screw. The lead screw in turn activates a plunger driver which forwardly pushes a plunger within a barrel of the syringe that has been removably installed in the pump. Pushing the plunger forward thus forces the infusate outwardly from the syringe, into the infusion line or tubing, and to the patient typically, intravenously. Examples of syringe pumps are disclosed in U.S. Pat. No. 4,978,335 titled “Infusion Pump with Bar Code Input to Computer,” U.S. Pat. No. 8,182,461 titled “Syringe Pump Rapid Occlusion Detection System,” and U.S. Pat. No. 8,209,060 titled “Updating Syringe Profiles for a Syringe Pump” and PCT Application Pub. No. WO2016/183342 titled “High Accuracy Syringe Pumps”. As used throughout this disclosure, the term “syringe pump” is intended to generally pertain to any device which acts on a syringe to controllably force infusates outwardly therefrom.

Syringe pumps are used to control the delivery of infusates to a patient that include, but are not limited to: therapeutic agents; nutrients; drugs; medicaments such as antibiotics, blood and blood products, coagulants, and analgesics; and other fluids. The devices can be used to introduce the infusates into patients'bodies utilizing any of several routes such as, for example, intravenously, subcutaneously, arterially, or epidurally.

It is sometimes desirable to remove the syringe from the syringe pump, manually administer a bolus to a patient, reinsert the syringe in the syringe pump, and resume delivery of infusate by the syringe pump. In this regard, boluses or loading doses can represent the largest volumes quickly delivered to patients. For example, an anesthesiologist may want to deliver a manual bolus dose of propofol to a patient by either: releasing a latch of the syringe pump's plunger driver head and advancing the plunger driver head manually to administer a bolus dose of the propofol from the syringe, or by entirely removing the syringe from the pump and manually pushing the plunger of the syringe to deliver the bolus. Currently, the volume of bolus delivered manually is visually estimated and recorded manually by a clinician (if the clinician remembers to record the visually estimated volume delivered, and with acceptable accuracy, and does not become incapacitated before recording it). The need to deliver the bolus manually may be to infuse at a faster rate than could be provided by the syringe pump, or to more quickly initiate the bolus without the time delay introduced by programming the bolus. The programmed infusion can currently be automatically captured electronically in a patient record, but the manual bolus is not since it takes place outside the pump-based infusion. There has therefore been a long-felt need to improve accuracy and reliability of the estimation and documentation of manual bolus administration by reducing or eliminating visual estimation of the volume and time of the manual bolus administered and subsequent manual data entry.

It would therefore be useful and advantageous to provide devices and methods for syringe pumps that calculate the estimate of the volume of a manual bolus administered while a syringe pump is not infusing, and that record and transmit such data to an electronic record-keeping system such as a Pharmacy Management System, an Electronic Medical Record (EMR) system, an Electronic Health Record (EHR) system, or a Hospital Information System (HIS) (collectively, an “Electronic System”).

### SUMMARY

This disclosure describes novel and inventive devices and methods for syringe pumps that calculate the volume of a manual bolus while delivery of infusate by a syringe pump is paused.

In the context of syringe pumps, infusate delivery from a syringe is typically directly proportional to movement of the plunger head of the syringe. Accordingly, embodiments described herein disclose devices and methods which measure plunger head position when delivery of infusate by a syringe pump is paused and again when delivery of infusate by a syringe pump is resumed. If the position of the plunger head is advanced more at the resumption of infusate delivery by the syringe pump than it was at the time delivery was paused, an estimate of the volume of a manual bolus given from the syringe temporarily removed from the syringe pump during administration of the manual bolus may be calculated and recorded by the syringe pump and/or by an Electronic System.

In an embodiment of the novel and inventive subject matter hereof, the syringe pump detects that one of its control features, such as defined field in a graphical user interface or a button, has been touched or pushed to cause a pause in syringe pump delivery of infusate. The syringe pump then records a first signal (e.g., of voltage or frequency) from a linear potentiometer within the syringe pump at that time. The first signal corresponds to a first position of the plunger driver of the syringe pump. After the manual bolus has been administered, the operator of the syringe pump reinserts the syringe into the syringe pump, slides the plunger driver forward and touches a defined field on the graphical user interface or a button to resume the infusion. The syringe pump records a second signal from the linear potentiometer at the time the syringe pump delivery of infusion is resumed. The second signal corresponds to a second position of the plunger driver of the syringe pump. The syringe pump then calculates, with respect to the first and second signals of the linear potentiometer, a distance that the plunger driver of the syringe pump has been moved forward and further calculates the volume displaced by multiplying the distance that the plunger driver has moved forward by the internal cross-sectional area of the syringe in use.

In an embodiment, the syringe pump can detect a feature of the syringe, such as an RFID tag or barcode portion of a syringe. The RFID tag or barcode, for example, can include information regarding a starting or initial volume of the syringe. The syringe pump can also or alternatively access information about the syringe through, for example, an Electronic System, including the inside diameter or volume per unit of syringe plunger displacement of the syringe. The syringe pump can also or alternatively calculate an estimate of the volume of the manual bolus, and record the volume of the manual bolus. In an embodiment, the syringe pump transmits its calculation of the bolus volume delivered per distance the plunger drive arm was advanced during the pause and the subsequent calculation of volume displaced to the Electronic System, to estimate and record the volume of the manual bolus. In an embodiment, the syringe pump displays a message on a user interface asking the operator of the syringe pump to confirm that a manual bolus had been administered during the pause in syringe pump delivery of infusate. In an embodiment, the syringe pump may prompt the operator to confirm the calculated manual bolus volume and/or dose when confirming the delivery of the manual bolus. In particular, the pump can prompt for confirmation that a manual bolus was delivered, and can also at that time present or display the bolus volume calculated for confirmation so that the operator can see and recognize the specific volume that will be recorded. If the operator confirms that a manual bolus was delivered, then the estimate is recorded by the syringe pump or the Electronic System.

In another embodiment, the volume of a manually-delivered bolus can be determined, or closely approximated, by alternate means of identifying the linear position of the syringe plunger, such as optical or other means of detection.

In another embodiment, the volume of a manually-delivered loading dose or induction dose, a bolus typically delivered at the start of an infusion prior to installing the syringe into a syringe pump, can be captured by the pump. In this embodiment, the loading dose volume can be calculated and recorded by considering an initial volume contained on a syringe label or recorded within an RFID tag on the syringe and a post-bolus volume calculated based on the syringe plunger position upon loading the syringe into the pump. Alternatively the loading dose could be delivered with the syringe installed in the pump by the clinician manually pushing the plunger assembly forward, in which case the linear position before and after the manual loading dose could be determined in the same manner as a manual bolus during an infusion.

Embodiments can include a syringe pump, including a pump housing and a drive assembly. The drive assembly can slideably move horizontally to extend and retract with respect to the pump housing and includes a plunger driver head, a drive arm, a lead screw, a motor, and a linear potentiometer. The plunger driver head has a surface structure that selectively and forcibly urges against a thumb press of a plunger of a syringe along a first central longitudinal axis when the syringe is installed in the syringe pump. The syringe is configured to contain an infusate and includes a barrel and the aforementioned plunger configured to slidably reside within the barrel and that together define the first central longitudinal axis. The drive arm includes an elongate plunger tube having a second longitudinal axis that is generally parallel to the first central longitudinal axis. The drive arm is coupled to the plunger driver head such that movement of the elongate plunger tube corresponds with generally equal and parallel movement of the plunger of the syringe along the first central longitudinal axis. The lead screw is operatively coupled to the drive arm and the motor is operatively coupled to the lead screw to govern movement of the lead screw and, consequently, the drive arm when the pump is operating to dispense an infusate from the syringe. The linear potentiometer generates a signal (e.g., of voltage or frequency) output proportional to an insertion distance of the rod inside the elongate plunger tube.

### BRIEF DESCRIPTION OF THE DRAWINGS

Devices for, and methods of, calculating an estimate of the volume of a manual bolus, administered while delivery of an infusate by a syringe pump is paused, are illustrated by way of example and not limitation in the figures of the accompanying drawings in which:

FIG. 1 is a front perspective view of a syringe pump, according to an embodiment.

FIG. 2 is a rear perspective view of the syringe pump of FIG. 1 according to an embodiment, permitting observation of interior components of a drive assembly that are generally obstructed by a housing of the syringe pump.

FIG. 3 is a partial perspective view of a syringe pump drive assembly, according to an embodiment.

FIG. 4 is a partial perspective view of a syringe pump drive assembly, according to an embodiment.

FIG. 5 is a partial side view of a syringe pump drive assembly, according to an embodiment.

FIG. 6 is an example of a linear potentiometer for use in a syringe pump drive assembly, according to an embodiment.

FIG. 7 is a flow chart of a method of calculating and recording an estimate of the volume of a manual bolus administered while delivery of infusate by a syringe pump is paused, according to an embodiment.

FIG. 8 is a flow chart of a method of calculating and recording an estimate of a volume of a manual loading dose, administered before reducing to a lower maintenance delivery of infusate by a syringe pump, according to an embodiment.

### DETAILED DESCRIPTION

Devices and methods described in greater detail by way of examples herein calculate and record an estimate of the volume of a manual bolus from a syringe administered while delivery of infusate by a syringe pump is paused such as when, for example, a manual bolus is given by a clinician to a patient after removal of the syringe from the pump. This can be accomplished by removing the syringe from the syringe pump, pushing the syringe plunger forward manually, reinserting the syringe into the syringe pump, and resuming delivery of infusate by the syringe pump. Such functionality can provide advantages not contemplated by existing syringe pump systems, as will be described by example herein.

In an example of an embodiment of a device for, and method of, calculating and recording an estimate of the volume of a manual bolus given by a clinician to a patient, the aforementioned functionality is achieved by way of a syringe pump that includes a drive assembly, or “drive train”, having a configuration enabling a linear potentiometer and corresponding capabilities. Embodiments of this arrangement contemplate enhanced documentation of the volume of infusate the patient has received at the time immediately after administration of the manual bolus and also the total volume of infusate administered to the patient at the time delivery of infusate by the syringe pump has been discontinued. Such documentation is an improvement to traditional or otherwise known processes in which the clinician visually estimates the volume of the manual bolus administered, and records it manually. During certain medical procedures and patient responses, the clinician may not have an adequate opportunity to accurately record the estimate and could err, for example, in later remembering the volume administered. Embodiments of this arrangement also contemplate reduction or elimination of a risk of human error that the manual bolus does not get entered into the Electronic System database, due to human forgetfulness, human incapacity, or loss of the documentation.

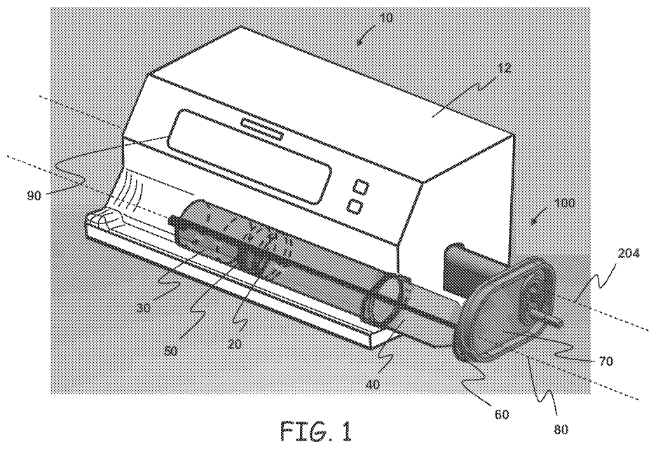

In general, accuracy and control of infusate delivery are two desired aspects of effective operation for a syringe pump. Based on the fundamental characteristics of its design, a syringe is intended to provide infusate delivery that is directly proportional to the movement of the drive arm. FIG. 1 illustrates an example of an embodiment of a syringe pump 10 that is configured for accurate infusate control. Syringe pump 10 includes a housing 12 configured to receive a syringe 20 having a barrel 30 and a plunger 40 with a plunger tip 50 and a thumb press 60. Housing 12 shown is largely box shaped and provides structure to surround interior drive components of pump 10 and for externally receiving syringe 20. Syringe 20 is typically prescribed by a pharmacist or other qualified clinician. Its inside diameter or its volume of delivery per unit length may be entered into an Electronic System. As an additional safeguard, the syringe may have a characteristic detectable by a syringe pump, such as a bar code or RFID tag, to allow confirmation by the syringe pump that the correct syringe was indeed loaded into the syringe pump. Syringe 20 is configured to contain a medication or other infusate to be delivered to a patient from syringe pump 10. Specifically, syringe pump 10 is configured to act on syringe 20 by way of a plunger driver head 70 being selectively and forcibly urged against thumb press 60 of plunger 40. Movement of plunger driver head 70 generally is controlled by a drive assembly 100 which permits slideable movement thereof horizontally to extend and retract with respect to housing 12. Barrel 30 and plunger 40 together define a central longitudinal axis 80 of syringe 20.

Syringe pump 10 also includes software (not explicitly illustrated) and display means that can, in an embodiment, be provided by way of suitable computing components (not explicitly illustrated) and a display screen or graphical user interface 90 located on, for example in FIG. 1, a front portion of syringe pump 10. Using interface 90, users can effectively observe and control, and otherwise interact with programming and operation of, pump 10. In some embodiments, such pump programming operations can interact with a infusate delivery “engine” that determines a volume of infusate being delivered from syringe 20, among other pump information and parameters, based in part on sensor data, as from a distance sensor, for example.

For purposes of this disclosure, the term “engine” can be defined as a real-world device, component, or arrangement of components implemented using hardware, or as a combination of hardware and software, such as by a processor system and a set of particular program instructions that adapt or prompt the engine to implement the particular functionality, which (while being executed) transform a processor system into a special-purpose device. An engine can also be implemented as a combination of the two, with certain functions facilitated by hardware alone, and other functions facilitated by a combination of software-controlled hardware. In certain implementations, at least a portion, and in some cases, all, of an engine can include the processor(s) of one or more computers that execute an operating system, system programs, and application programs, while also implementing the engine using multitasking, multithreading, distributed (e.g., cluster, peer-peer, cloud, etc.) processing where appropriate, or other such techniques. In addition, an engine can itself be composed of more than one sub-engines, each of which can be regarded as an engine, whether collectively or individually.

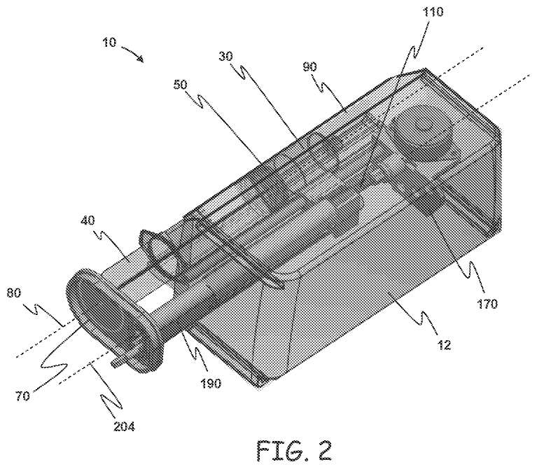

Referring now to FIGS. 2-5, and with continued reference to FIG. 1, therein illustrated are various partial views of embodiments of a drive assembly 100 of syringe pump 10. Drive assembly 100 of pump 10 includes a plunger driver head 70, a linear potentiometer 110, plunger tube 120, lead screw 130, lead screw housing 140, gears 150, and motor 160. In general, drive assembly 100 is supported between a first support plate 170 and a second support plate 180, respectively located at opposite sides of housing 12. Extending through second support plate 180 is a plunger head drive arm 190. Plunger head drive arm 190 generally refers to a combination of elongate components extending across drive assembly 100 and can include plunger tube 120 and a lead screw housing 140, for example. Plunger tube 120 can comprise a hollow tube, extending across a top portion of plunger head drive arm 190, but may also be integrally formed with lead screw housing 140 in some embodiments as well.

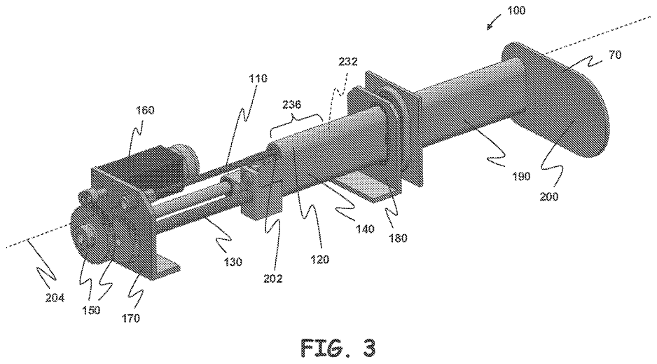

Plunger driver head 70 is shown in FIG. 3 as a plate or generally oblong disc-like component that is attached near one edge of its surface to an end of plunger head drive arm 190. With reference also to FIG. 1, an opposite edge of plunger driver head 70 provides a surface structure 200 (as identified in FIG. 3) to selectively engage and forcibly urge thumb press 60 of syringe 20 along longitudinal axis 80. This surface structure 200 can simply include a generally flat surface of plunger driver head 70 or can include various projections or recessed areas for providing engagement with thumb press 60 when syringe 20 is installed in pump 10. Although not illustrated, driver head 70 can include one or more thumb press capture components or “flippers”, that releasably engage the syringe's thumb press flange or other capturable surface. Accordingly, plunger driver head 70 is able to controllably push thumb press 60, and thus plunger 40 within barrel 30 of syringe 20, in response to turning by lead screw 130 that consequently draw plunger driver head 70 toward housing 10. Turning of lead screw 130, accordingly, is controlled by rotational motion provided by motor 160 which drives gears 150 that engage and enable rotational movement of lead screw 130.

As shown most visibly in FIGS. 2-4, linear potentiometer 110 extends between first support plate 170 and plunger tube 120. Specifically in this example embodiment, linear potentiometer 110 is secured to first support plate 170 at an end thereof and extends into the aperture opening 202 of plunger tube 120 of plunger head drive arm 190 at an opposite end. The elongate plunger tube 120 has a longitudinal axis 204 through its center that is generally parallel to longitudinal axis 80 (as shown in, e.g., FIG. 2) and which is in alignment with a longitudinal axis of linear potentiometer 110. Further, linear potentiometer 110 is located in a substantially parallel orientation with central longitudinal axis 80 of syringe 20 when installed in syringe pump 10. Likewise in this example embodiment (as shown in, e.g., FIG. 3), lead screw 130 and motor 160 are secured to first support plate 170. Lead screw 130 is located in a substantially parallel orientation with central longitudinal axis 80 of syringe 20 and linear potentiometer 110 as well. Embodiments use a linear potentiometer 110 that is an accurate type of linear potentiometer which is specifically integrated into a syringe pump 10.

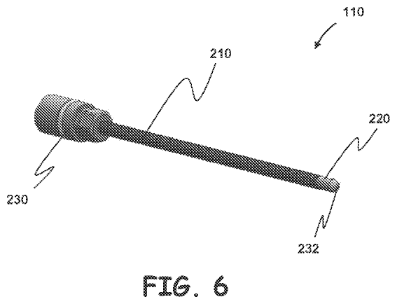

In certain embodiments, the linear potentiometer 110 includes, for example, an inner wrapped coil which causes current to flow in a clockwise direction and an outer wrapped coil which causes current to flow in a counterclockwise direction. For example, possible linear potentiometers that could be used in embodiments of the proposed syringe pumps include DIST (Distributed Impedance Sensor Technology) sensors and related technology manufactured by LRT Sensors, LLC. of Huntingdon Valley, PA. In such embodiments, as shown in FIG. 6, linear potentiometer 110 can be a sensor that comprises a double coil 210 wound on a rounded non-conductive fiberglass rod 220. The wire is wound in a first coil as a helix having a large pitch. At the end of rod 220, the pitch is reversed and a second coil as a returning helix is laid over the first coil. An end of rod 220 comprises electronics 230 including a transistor connected to a resulting helical wire assembly from the first and second coils that produce a resonant circuit and oscillation. The opposite end of rod 220 comprises rod tip 232, which is the portion of rod 220 that is first concealed by plunger tube 120 during pump operation.

This arrangement results in two coils in series with one having a generally clockwise current flow and the other having a generally counterclockwise current flow. When energized, resulting magnetic fields of the coils are generally parallel to the sensor, but in opposite directions, and accordingly cancel out each other. Electric fields from the currents are generally perpendicular to rod 220 and accordingly are additive. This provides an electromagnetic field outside the coils that is largely electric. Consequently, the exemplary linear potentiometer 110 discussed requires no magnets or magnetic material in various embodiments. Potentiometer 110 is generally insensitive to external magnetic fields and may accordingly permit satisfactory operation in environments subject to high magnetic fields.

A frequency of the circuit containing a DIST sensor is determined by inductance and capacitance of the helical wire assembly. In general, operating a circuit at its resonant frequency produces a very stable output. The inductance of the assembly is low and constant, due to relatively few turns of the first and second coils, but a ratio of capacitance to inductance is higher than in an inductive sensor and is based on an interaction of a strong electric field of the sensor with any nearby conductive surface. Accordingly, one way in which to cause capacitance and resonant frequency of the sensing element to change significantly is to cover the coil and rod with a conductive structure. In embodiments of syringe pump 10 containing a DIST sensor, the helical wire assembly and rod 220 are partially and slideably covered by elongate plunger tube 120 made of conductive material. Even one MHz change in resonant frequency can result when plunger tube 120 moves over potentiometer 110. Changes in frequency are linear with movement of plunger tube 120. These changes can be transmitted and converted to digital signals for further processing as desired.

A DIST sensor used as a linear potentiometer 110 is advantageous as it is a relatively simple device requiring a single wire for power and transmission of information. Digital output frequency can be “piggybacked” or carried on or with DC power so that only one wire is required and the receiver can be located remotely. This wire provides great flexibility in locating signal analysis electronics based in part on its significant length of signal wire. In general, problems are minimized by only using one wire. Accordingly, a DIST sensor is a simple, economical and compact device that can help make accurate linear measurements with only a single wire for power and signal.

DIST sensors do not require use of extremely fine wire as in other sensor constructions and can instead use a heavy wire since it is generally designed to maximize capacitance. A more robust wire is advantageous as a heavy wire wound on a flexible rod can withstand extremely high levels of shock and vibration and overcomes these susceptibilities of other sensors.

In some embodiments, a traditional linear potentiometer may be used instead of a DIST sensor. The resistance to electrical current run through a traditional linear potentiometer in a syringe pump is indicative of the position of the plunger rod drive arm, such that the voltage output is indicative of its linear position. An example of a commercialized pump with such a traditional linear potentiometer is a MEDFUSION 4000 syringe pump manufactured by Smiths Medical ASD, Inc.

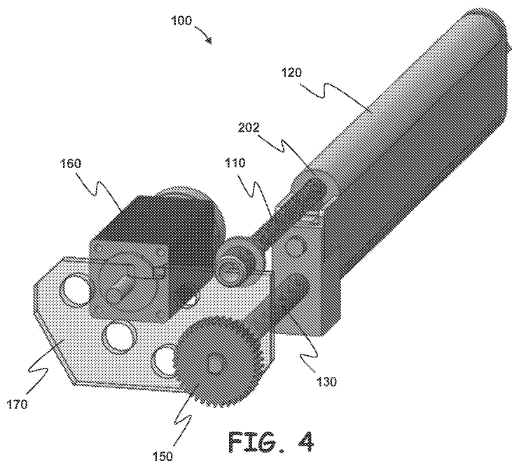

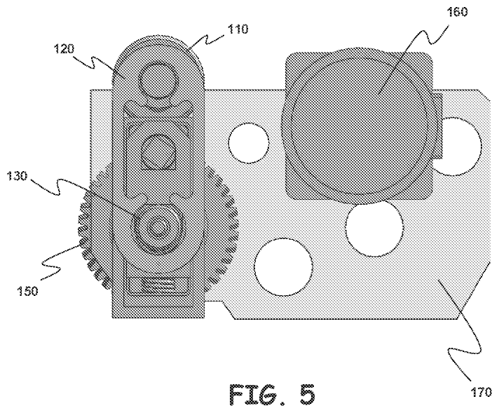

FIGS. 4 and 5 further show partial views of embodiments of syringe pump drive assembly 100 in which a structural relationship and arrangement of components permitting effective use of a type of linear potentiometer such as potentiometer 110 can be more fully understood. Specifically, in these embodiments, linear potentiometer 110 and plunger tube 120 are axially aligned with one another. Further, linear potentiometer 110 and plunger tube 120 are vertically located above lead screw 130. Accordingly, respective axes of rod 220 of linear potentiometer 110, elongate plunger tube 120, and lead screw 130 are generally parallel to each other and project outwardly from the generally flat surface of first support plate 170 in a generally perpendicular orientation.

Accordingly, and with reference also to FIG. 3, linear potentiometer 110 can be a DIST sensor that generates a frequency output that is proportional to a distance of insertion 236 of the sensor (i.e. rod 220) inside a metal or aluminum tubing such as plunger tube 120. This insertion distance 236 of the sensor can be understood to represent the covered length of rod 220 extending between the aperture opening 202 of the plunger tube 120 and the location of rod tip 232 within the plunger tube 120. This insertion distance 236 of the sensor (i.e. rod 220) determines frequency output. Accordingly, as a location of plunger tube 120—relative to, for example, plate 170—changes when it slidingly moves with drive arm 190, the frequency output also changes. This frequency can be measured to within one Hz in various embodiments. A corresponding change in distance for plunger movement for such a one Hz measurement would be roughly one micron in certain embodiments. Such a level of accuracy allows for detection of displacement of within one micron, whereas previously known syringe pumps may be capable of a resolution of within only about 500 microns. In other embodiments, a traditional linear potentiometer may be used instead of a DIST sensor.

In general, therefore, it is to be understood that the linear potentiometer and associated electronics and processing hardware/software of an infusate delivery engine, as described by example or otherwise contemplated herein for syringe pump 10, enable determination of specific positions of a plunger relative to a barrel of a syringe installed in pump 10. In certain embodiments, linear potentiometers may include position sensing technology similar to that described in U.S. Pat. No. 7,216,054 to Pchelnikov et al. and in U.S. Pat. No. 8,692,541 to Nyce et al. Accordingly, linear potentiometer 110, such as embodied in an aforementioned DIST sensor, is integrated into an architecture of drive assembly 100 of syringe pump 10. The architecture must be constructed to support an arrangement in which frequency output is proportional to a displacement of linear potentiometer 110 inside plunger tube 120. As depicted in FIGS. 2-5, some embodiments are accordingly constructed such that lead screw 130 that drives movement of thumb press 60, and consequently, plunger 40, are located in a generally parallel arrangement such that movement of plunger 40 will directly correspond to movement of potentiometer 110 and result in detection of resulting frequency output signals. Accordingly, movement of plunger 40 along longitudinal axis 80 moves plunger tube 120 an approximately equal distance in the same direction. Further, linear potentiometer 110 is located such that it is firmly coupled to first support plate 170 at its end. Lead screw 130 is coupled to a stepper motor 160 via gears 150 that may be housed in a gear box (not shown). Lead screw 130 drives plunger tube 120 in relation to linear potentiometer 110 as well as lead screw housing 140. Both plunger tube 120 and lead screw housing 140 comprise components of plunger head drive arm 190. Therefore, motor 160 is operatively coupled to lead screw 130 to govern movement of lead screw 130 and, therefore also, drive arm 190.

Consequently, infusate delivery and determination occurs when plunger head drive arm 190 acts to advance plunger 40 within syringe 20. Drive arm 190 causes linear potentiometer 110 to sense a present position of drive arm 190, by measuring, for example, frequency or voltage output as previously described. With a position of drive arm 190 thus known, a location of plunger tip 50 is determined and the software can accurately provide valuable information on delivery of infusate from the syringe by the pump.

Display screen or graphical user interface 90 can provide infusate delivery information and controls for syringe pump 10. Interface 90 can comprise, for example, suitable touch screen or LCD technology. Examples of touch screen devices generally are disclosed in U.S. Pat. Applic. Pub. Nos. 2006/0097991 titled “Multipoint Touchscreen” and in 2011/0193788 titled “Graphical Objects that Respond to Touch or Motion Input.” Examples of novel and inventive infusion pump technologies employing touch screen devices are disclosed in: U.S. Pat. No. 5,485,408 titled “Pump Simulation Apparatus”; U.S. Pat. Applic. Pub. No. 2009/0270810 titled “Security Features for a Medical Infusion Pump.”

It is also to be appreciated and understood that types, components, dimensions, fabrication processes, and other particulars and parameters of aforedescribed example embodiments can be substituted for others as desired, or that accessories can be added thereto.

It is again to be appreciated and understood that during administration of infusate by a clinician to a patient, the clinician may pause delivery of infusate by the syringe pump, remove the syringe from the syringe pump, manually advance the plunger of the syringe to administer a “manual bolus”, reinsert the syringe into the syringe pump, and cause the syringe pump to resume delivery of infusate. An estimate of the volume of a manual bolus given from the syringe 20 temporarily removed from a syringe pump 10 during administration of the manual bolus may be calculated and recorded by the syringe pump 10. The syringe pump 10 detects that one of its control features, such as defined field in a graphical user interface 90 or a button, has been touched or pushed to cause a pause syringe pump delivery of infusate. The syringe pump then records, for example, the frequency or voltage signal from the linear potentiometer 110 at that time. After the manual bolus has been administered, the operator of the syringe pump 10 reinserts the syringe 20 into the syringe pump 10, slides the plunger head drive arm 190 forward and touches a defined field on the graphical user interface 90 or a button to resume delivery of infusate by the syringe pump. The syringe pump 10 records the signal from the linear potentiometer 110 at the time the syringe pump delivery of infusion is resumed. The syringe pump 10 then calculates the distance that the plunger head drive arm 190 has been moved forward during the time that delivery of infusate by syringe pump 10 was paused.

The syringe pump 10 may detect a feature of the syringe 20; such as an RFID tag or barcode portion of a syringe 20; access information about the syringe 20 though an Electronic System, including the inside diameter or volume per unit of plunger displacement of the syringe 20; calculate an estimate of the volume of the manual bolus; and record the estimate of the volume of the manual bolus. In an embodiment, the syringe pump 10 then transmits the information to the Electronic System for recording. In an embodiment, the syringe pump 10 need not detect a feature of the syringe 20 and transmits its calculation of the distance the plunger drive arm 190 was advanced during the pause to the Electronic System and the Electronic System calculates and records the estimate of the volume of the manual bolus.

In an embodiment, the syringe pump 10 displays a message on the interface 90 asking the operator of the syringe pump 10 to confirm that a manual bolus had been administered during the pause in delivery of infusate by the syringe pump. In an embodiment, the volume and/or dose calculated is presented to the clinician in the confirmation. If the operator confirms that a manual bolus was administered, only then is the estimate of the manual bolus recorded.

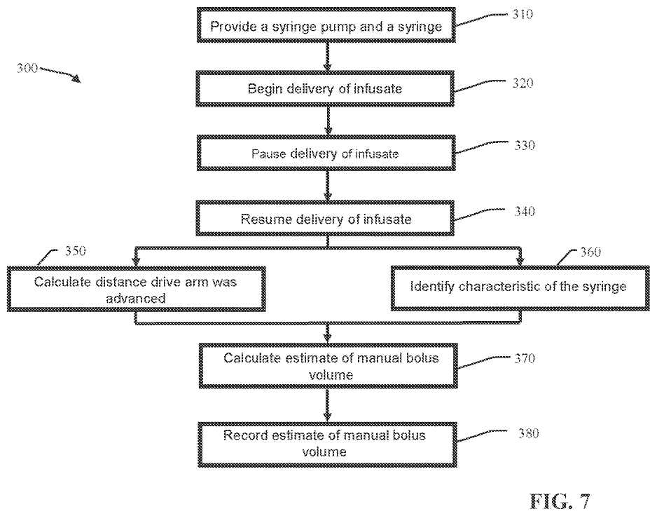

Now referring to FIG. 7, an example of operation of a syringe pump system including a method of calculating and recording an estimate of a manual bolus is illustrated. In FIG. 7, an embodiment described by a flow chart of a method 300 of calculating and recording an estimate of a manual bolus is disclosed. First, at 310 a syringe pump such as pump 10 is provided that includes a pump housing 12 and a drive assembly 100 that slideably extends and retracts with respect to the pump housing 12. The drive assembly 100 includes a plunger driver head 70, a drive arm 190, a lead screw 130, a motor 160 and a linear potentiometer 130. Further, the method at 310 includes receiving syringe 20 in syringe pump 10 filled with a total volume of an infusate.

At 320, the method also includes beginning delivery of infusate by syringe pump 10. At 330 the method further includes pausing delivery of infusate by syringe pump 10.

At 340 the method further includes resuming delivery of infusate by syringe pump 10. At 350, syringe pump 10 calculates the distance that the plunger rod drive arm was advanced while delivery of infusate by syringe pump 10 was paused by comparing the signal from linear potentiometer 110 at the time delivery of infusate by syringe pump 10 was paused to the signal from linear potentiometer 110 at the time delivery of infusate by syringe pump 10 was resumed.

At 360, the syringe pump 10 or the Electronic System determines a characteristic of the syringe indicative of its inside diameter or volume per unit of syringe plunger displacement. At 370, the syringe pump 10, the Electronic System, or both calculate an estimate of the volume of the manual bolus (such as, for example, administered by a clinician to a patient). At 380, the syringe pump 10, the Electronic System, or both record the estimate of the volume of the manual bolus.

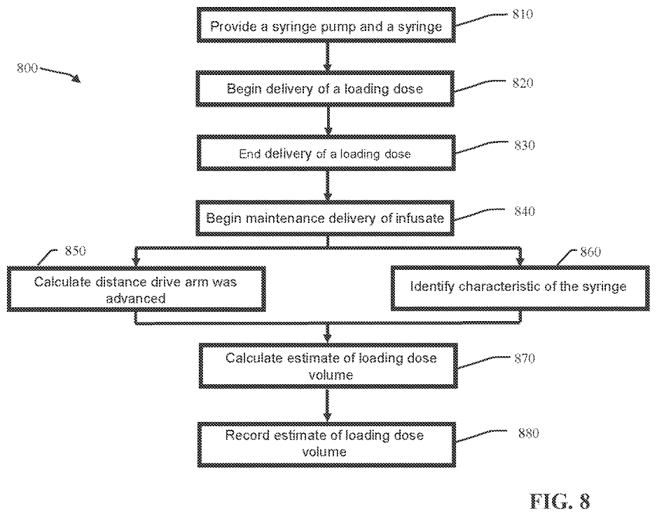

Referring now to FIG. 8, an example of operation of a syringe pump system including a method of calculating and recording an estimate of a manual loading dose or induction dose (“loading dose”) is illustrated. A loading dose of an infusate may be administered, without having to rely on the pump for its administration, when for example an initial higher dose of the infusate is necessary or desired to be infused relatively quickly, at the beginning of a process of an infusion to a patient, before reducing to a lower maintenance delivery of the infusate.

In FIG. 8, an embodiment described by a flow chart of a method 800 of calculating and recording an estimate of a loading dose is disclosed. First, at 810 a syringe pump such as pump 10 is provided that includes a pump housing 12 and a drive assembly 100 that slideably extends and retracts with respect to the pump housing 12. The drive assembly 100 includes a plunger driver head 70, a drive arm 190, a lead screw 130, a motor 160 and a linear potentiometer 130. Further, the method at 810 includes receiving syringe 20 in syringe pump 10 filled with a total volume of an infusate.

At 820, the method also includes beginning delivery of a loading dose by, for example, disengaging syringe 20 from plunger driver head 70 and manually advancing the plunger of syringe 20, corresponding to the volume or amount of the intended loading dose or an observed patient response. At 830 the method further includes ending delivery of the loading dose.

At 840 the method further includes beginning a lower maintenance delivery of infusate by syringe pump 10 by, accordingly, re-engaging the plunger of syringe 20 with plunger driver head 70. At 850, syringe pump 10 calculates the distance that the plunger rod drive arm was advanced from delivery of the loading dose by comparing the signal from linear potentiometer 110 at the time just before delivery of the loading dose to the signal from linear potentiometer 110 at the time when the lower maintenance delivery of infusate began.

At 860, the syringe pump 10 or the Electronic System determines a characteristic of the syringe indicative of its inside diameter or volume per unit of syringe plunger displacement. At 870, the syringe pump 10, the Electronic System, or both calculate an estimate of the volume of the loading dose. At 880, the syringe pump 10, the Electronic System, or both record the estimate of the volume of the loading dose.

In an embodiment, rather than removing the syringe from the pump to manually deliver the loading dose, the clinician could leave the syringe mounted in the pump and manually deliver the loading dose by manually pushing forward the pump plunger driver head 70 (FIG. 1) which simultaneously pushes forward mounted syringe plunger 40 into barrel 30 to deliver the loading dose to the patient.

Although not specifically illustrated, it is to be appreciated and understood that processes described with reference by example to FIGS. 7 and 8 could be provided alternatively or in any suitable combination with each other to advantageously result in efficiently estimating and/or recording a particular infusate delivery to a patient.

It is also to be appreciated and understood that subject matter hereof is suitable for and can accommodate various infusion measurements or parameters, depending on particular clinical needs. For example, a manual bolus or a loading dose may be characterized as a volume (e.g., 5 mL) or as a dose (e.g., 5 mg).

Irrespective of a particular embodiment, it is to be appreciated and understood that in embodiments of devices, systems, and methods for syringe pumps that estimate and record the volume of a manual bolus provide convenience and efficiency, and reduces a likelihood that the volume of the manual bolus might not be documented accurately or at all.

It is also to be appreciated and understood that although reference has been made herein to manual bolus administration to a patient, embodiments of devices, systems, and methods—as described by example or otherwise contemplated herein—can be useful for virtually any situation where a syringe is removed from a syringe pump and the syringe plunger is manually advanced before reinstalling the syringe in the pump. Such a situation may be, for example, when air is visible in the syringe and needs to be quickly expelled or when a “flush” of infusate is desired, through tubing connected to the syringe.

It is further to be appreciated and understood that in some situations a manual bolus may be delivered when the pump is either infusing or paused. For example, an anesthesiologist may be directing a continuous infusion of propofol but a patient receiving the infusion prematurely begins to come out of that anesthesia's effect. In such a situation the anesthesiologist might quickly direct administration of a “top-up” bolus to keep the patient adequately under the anesthesia's effect. Devices, systems, and methods that have been described by example or otherwise contemplated herein can thus advantageously provide estimation and recording of such a “top-up” bolus.

It is additionally to be appreciated and understood that information exchange or code recognition can be advantageously employed in devices, systems, and methods that have been described by example or otherwise contemplated herein. For example, an information exchange or code recognition system, such as RFID or barcode (as aforementioned), or QR code, could be useful in situations when a syringe is completely removed from a pump and the operator is then uncertain about whether the same syringe was reinstalled in the pump or desires affirmative confirmation thereof. The information exchange or code recognition system (e.g., RFID, barcode, or QR code as aforementioned) could be on or with the syringe, to assist in verification that the syringe was not inadvertently exchanged with another syringe in similar brand and/or capacity, or with another syringe containing a different medication, or with another syringe containing the same medication with a different concentration.

While devices, systems, and methods for syringe pumps that estimate and record the volume of a manual bolus have been particularly shown and described with reference to the accompanying figures and specification, it should be understood, however, that other modifications thereto are of course possible; and all are intended to be within the true spirit and scope of novel and inventive devices, systems, and methods described herein. Thus, configurations and components of various features could be modified or altered depending upon particular embodiments. For example, additional steps could be included in the various method steps described by example or otherwise contemplated herein, such as the syringe pump displaying a message asking the operator of the syringe pump to confirm that a manual bolus was administered.

It is also to be understood in general that any suitable alternatives may be employed to provide novel and inventive devices, systems, and methods for syringe pumps that estimate and record the volume of a manual bolus that have been described by example or are otherwise contemplated herein. Compositions, sizes, and strengths of various aforementioned components of devices, systems, and methods for syringe pumps that estimate and record the volume of a manual bolus that have been described by example or are otherwise contemplated herein are all a matter of technical choice depending upon intended uses thereof.

Accordingly, these and other various changes or modifications in form and detail may also be made, without departing from the true spirit and scope of devices, systems, and methods for syringe pumps that estimate and record the volume of a manual bolus.

1. A syringe pump, comprising:
a pump housing;
a drive assembly that slideably extends and retracts with respect to the pump housing, including:
a plunger driver head having a surface structure that selectively urges against a syringe along a first central longitudinal axis when the syringe is installed in the syringe pump, wherein the syringe is configured to contain an infusate and includes a barrel and a plunger that define the first central longitudinal axis;
a drive arm, including an elongate plunger tube having a second longitudinal axis that is generally parallel to the first central longitudinal axis, coupled to the plunger driver head such that movement of the elongate plunger tube corresponds with substantially equal and substantially parallel movement of the plunger of the syringe along the first central longitudinal axis;
a lead screw operatively coupled to the drive arm;
a motor operatively coupled to the lead screw for movement of the lead screw and the drive arm;
a potentiometer, the potentiometer generating a signal that is proportional to an insertion distance of a rod inside the elongate plunger tube;
a control feature for use by an operator of the syringe pump to pause delivery of infusate by the syringe pump;
a control feature for use by the operator of the syringe pump to resume delivery of infusate by the syringe pump; and
a processor that calculates the distance that the drive arm was advanced while the delivery of infusate by the syringe pump was paused.

2. The syringe pump of claim 1, wherein the syringe pump detects information about the syringe to identify a characteristic of the syringe, including an inside diameter dimension or volume per unit of syringe plunger displacement, and calculates the volume of a manual bolus performed while delivery of infusate by the syringe pump was paused.

3. The syringe pump of claim 2, wherein the syringe pump records the volume of the manual bolus given while delivery of infusate by the syringe pump was paused.

4. The syringe pump of claim 2, wherein the syringe pump transmits the volume of the manual bolus given while delivery of infusate by the syringe pump was paused to an electronic system.

5. The syringe pump of claim 2, wherein the syringe pump transmits the distance the drive arm was advanced while delivery of infusate by the syringe pump was paused to an electronic system, and the electronic system calculates the volume of the manual bolus performed while delivery of infusate by the syringe pump was paused.

6. A method of calculating the volume of a manual bolus performed while delivery of infusate by a syringe pump is paused, comprising:
providing a syringe pump, including:
a pump housing;
a drive assembly that slideably extends and retracts with respect to the pump housing, including:
a plunger driver head having a surface structure that selectively urges against a syringe along a first central longitudinal axis when the syringe is installed in the syringe pump, wherein the syringe is configured to contain an infusate and includes a barrel and a plunger that define the first central longitudinal axis;
a drive arm, including an elongate plunger tube having a second longitudinal axis that is substantially parallel to the first central longitudinal axis, coupled to the plunger driver head such that movement of the elongate plunger tube corresponds with substantially equal and parallel movement of the plunger of the syringe along the first central longitudinal axis;
a lead screw operatively coupled to the drive arm;
a motor operatively coupled to the lead screw for movement of the lead screw and the drive arm;
a potentiometer, the potentiometer generating a signal that is proportional to an insertion distance of the rod inside the elongate plunger tube;
a control feature for use by the operator of the syringe pump to pause delivery of infusate by the syringe pump;
a control feature for use by the operator of the syringe pump to resume delivery of infusate by the syringe pump;
a processor that calculates the distance that the drive arm was advanced while the delivery of infusate by the syringe pump was paused; and
calculating the distance that the drive arm was advanced while delivery of infusate by the syringe pump was paused.

7. The method of claim 6, wherein the syringe pump detects information about the syringe to identify a characteristic of the syringe, including an inside diameter dimension or volume per unit of syringe plunger displacement, and calculates the volume of the manual bolus performed while delivery of infusate by the syringe pump was paused.

8. The method of claim 7, wherein the syringe pump records the volume of the manual bolus given while delivery of infusate by the syringe pump was paused.

9. The method of claim 6, wherein the syringe pump transmits the volume of the manual bolus given while delivery of infusate by the syringe pump was paused to an electronic system.

10. The method of claim 7, wherein the syringe pump transmits the distance the drive arm was advanced while delivery of infusate by the syringe pump was paused to an electronic system, and the electronic system calculates the volume of the manual bolus given while delivery of infusate by the syringe pump was paused.

11-14. (canceled)

15. A syringe pump, comprising:
a drive assembly, including:
a plunger driver head configured to urge against a syringe when the syringe is installed in the syringe pump, the syringe configured to contain an infusate and includes a barrel and a plunger defining a first longitudinal axis;
a drive arm, including an elongate plunger tube having a second longitudinal axis, the drive arm coupled to the plunger driver head such that movement of the elongate plunger tube corresponds with substantially equal and substantially parallel movement of the plunger of the syringe along the first longitudinal axis;
a control feature for use by an operator of the syringe pump to pause or resume delivery of infusate by the syringe pump; and
a processor that calculates the distance that the drive arm was advanced while the delivery of infusate by the syringe pump was paused.

16. The syringe pump of claim 15, further comprising:
a lead screw operatively coupled to the drive arm; and
a motor operatively coupled to the lead screw for movement of the lead screw and the drive arm

17. The syringe pump of claim 16, further comprising:
a potentiometer, the potentiometer generating a signal that is proportional to an insertion distance of a rod inside the elongate plunger tube.

18. The syringe pump of claim 17, wherein the syringe pump is configured to record the signal from the potentiometer at a time when delivery is paused.

19. The syringe pump of claim 18, wherein the syringe pump is configured to record the signal from the potentiometer at a time when delivery is resumed.

20. The syringe pump of claim 19, wherein the control feature is configured to display on a user interface a message requesting the operator confirm a manual bolus had been administered during the time when delivery was paused.
